(19) United States
(12) Patent Application Publication
(10) Pub. No.: US 2024/0180834 A1
(43) Pub. Date: Jun. 6, 2024

(54) DEVELOPMENT OF COVID-19 VACCINE USING A DUAL TLR LIGAND LIPOSOME ADJUVANT

(71) Applicants: Christopher Fox, Sumner, WA (US); Anna Pomés, Charlottesville, VA (US); Univesity of Virginia Patent Foundation, Charlottesville, VA (US)

(72) Inventors: William A. Petri, Jr., Charlottesville, VA (US); Mayuresh M. Abhyankar, Charlottesville, VA (US); Barbara J. Mann, Charlottesville, VA (US); Peter Kasson, Charlottesville, VA (US); Anna Pomés, Charlottesville, VA (US); Christopher Fox, Sumner, WA (US)

(21) Appl. No.: 18/279,193

(22) PCT Filed: Feb. 28, 2022
(86) PCT No.: PCT/US2022/018188

§ 371 Date: Aug. 28, 2023

Related U.S. Application Data
(60) Provisional application No. 63/154,469, filed on Feb. 26, 2021.

(51) Int. Cl. A61K 9/127; A61K 9/00; A61K 9/19; A61K 39/00; A61K 39/215; A61K 39/39

(52) U.S. Cl. CPC A61K 9/1271; A61K 9/0019; A61K 9/0043; A61K 9/19; A61K 39/215; A61K 39/39; A61K 2039/55555; A61K 2039/575; C12N 2770/20034

(57) ABSTRACT

Disclosed are compositions for eliciting anti-SARS-CoV-2 immune responses in subjects. In some embodiments, the compositions include one or more SARS-CoV-2 antigens and one or more PEGylated liposomal adjuvants, wherein at least one of the PEGylated liposomal adjuvants includes a cholesterol, a non-PEGylated neutral lipid, and a PEGylated lipid. Also provided are methods for using the presently disclosed compositions for stimulating anti-SARS-CoV-2 immune responses, for inducing anti-SARS-CoV-2 Th1 responses, for stimulating systemic immune responses and/or mucosal immune responses, for inducing anti-SARS-CoV-2 IgA responses, for reducing SARS-CoV-2-induced lung injuries, and for inducing anti-SARS-CoV-2 neutralizing antibodies in subjects in need thereof.

### CROSS REFERENCE TO RELATED APPLICATION

The presently disclosed subject matter claims the benefit of U.S. Provisional Patent Application Ser. No. 63/154,469, filed Feb. 26, 2021, the disclosure of which incorporated herein by reference in its entirety.

### GOVERNMENT INTEREST

This invention was made with government support under Grant No. 272201800025C-P00001-9999-1 awarded by The National Institutes of Health. The government has certain rights in the invention.

### REFERENCE TO SEQUENCE LISTING SUBMITTED ELECTRONICALLY

The content of the electronically submitted sequence listing in ASCII text file (Name: 3062_152_PCT_ST25.txt; Size: 32 kilobytes; and Date of Creation: Feb. 28, 2022) filed with the instant application is incorporated herein by reference in its entirety.

### TECHNICAL FIELD

The presently disclosed subject matter relates in some embodiments to compositions and methods for stimulating anti-SARS-CoV-2 immune responses, inducing anti-SARS-CoV-2 Th1 responses, stimulating systemic immune responses and mucosal immune responses, inducing anti-SARS-CoV-2 IgA responses, reducing SARS-CoV-2-induced lung injuries, and for inducing anti-SARS-CoV-2 neutralizing antibodies in subjects in need thereof.

### BACKGROUND

Respiratory tract infections remain the top cause of morbidity and mortality from infectious diseases worldwide. The explosive outbreak of severe acute respiratory syndrome coronavirus 2 (SARS-CoV-2) that led to the COVID-19 pandemic underlines a persistent threat of respiratory tract infectious diseases and warrants preparedness for a rapid response. COVID-19 is primarily a lower respiratory tract infection that can result in flu or pneumonia-like symptoms. Severe disease can manifest as an acute respiratory distress syndrome (ARDS), multi-organ failure, cytokine storm, or death (Huang et al., 2020). Numerous studies characterizing the immune response in COVID-19 patients have revealed more disparities than commonalities (Desai et al., 2020; Manson et al., 2020; Chia et al., 2021; Teijaro & Farber, 2021). According to data complied by the World Health Organization, over one hundred vaccines based on different technologies have been being assessed in various phases of clinical development and twenty vaccines had been authorized for emergency use worldwide by various regulatory authorities. The emergence of more transmissible variants, unavailability of data on the longevity of response, the effect of age, and co-morbidities on response to the vaccine are additional knowledge gaps that need to be addressed. It is therefore imperative to explore various vaccine platforms and strategies in parallel (Jeyanathan et al., 2020)

SARS-CoV-2 is a single-stranded positive-strand RNA virus and spike protein is the primary target of neutralizing antibodies (Hodgson et al., 2021) An effective vaccine should induce high titers of neutralizing antibodies preferably with minimum antigen amount and doses. Incorporating a suitable adjuvant in a vaccine may address these requirements. Arunachalam et al. recently evaluated the potential of adjuvanted SARS-CoV-2 spike protein receptor-binding domain (RBD) to elicit the neutralizing response in monkeys. Although all the five adjuvants tested induced substantial neutralizing antibody titers, different profiles of Th1-Th2 responses as well as varying levels of protection against SARS-CoV-2 were observed depending upon the adjuvant platform (Arunachalam et al., 2021). Toll-like receptors (TLRs) are a category of pattern-recognition receptors critical for pathogen recognition. TLR agonists have been extensively studied as vaccine adjuvants as they allow rapid activation of innate immunity, and subsequently, effective adaptive immunity (Luchner et al., 2021).

We have recently developed a fully synthetic dual TLR nanoliposome adjuvant that can concurrently elicit antigen-specific mucosal and systemic responses (Abhyankar et al., 2017; Abhyankar et al., 2018). The TLR4 agonist GLA favored a mucosal IgA response whereas TLR 7/8 agonist 3M-052 elicited a robust systemic Th1 response. This adjuvant can be used for mucosal immunization, and an intranasal route of administration was found to be important for the generation of these immune responses (Abhyankar et al., 2018). Based on these observations, we tested the protective capability of adjuvanted Spike vaccine using the SARS-CoV-2 K18-hACE2 mouse infection model (Moreau et al., 2020).

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to compositions for eliciting anti-SARS-CoV-2 immune responses in subjects in need thereof. In some embodiments, the compositions comprise, consist essentially of, or consist of a SARS-CoV-2 antigen and a PEGylated liposomal adjuvant, wherein the PEGylated liposomal adjuvant comprises, consists essentially of, or consists of a cholesterol, a non-PEGylated neutral lipid, and a PEGylated lipid. In some embodiments, the average molecular weight of the PEG in the PEGylated lipid is about 5000 Daltons or less. In some embodiments, the average molecular weight of the PEG in the PEGylated lipid ranges from about 750 Daltons to about 5000 Daltons, optionally about from about 750 Daltons to 2000 Daltons. In some embodiments, the average molecular weight of the PEG in the PEGylated lipid is about 2000 Daltons or less. In some embodiments, the lipid component of the PEGylated lipid comprises a neutral lipid. In some embodiments, the lipid component of the PEGylated lipid is DSPE, DPPC, DOPC, DLPC, DMPC, DSPC, POPC, DPPE, or DMPE. In some embodiments, the lipid component of the PEGylated lipid is DSPE or DPPE. In some embodiments, the non-PEGylated neutral lipid is DPPC, DOPC, DLPC, DMPC, DSPC, POPC, DPPE, or DMPE. In some embodiments, the non-PEGylated neutral lipid is DPPC. In some embodiments, the liposome is stable for at least 1 month at a temperature of about 2° C. to about 8° C. In some embodiments, the polydispersity index of the liposome is maintained at about 0.3 or less. In some embodiments, the size of the liposome less than about 450 nm. In some embodiments, the molar percentage (mol %) of the PEGylated lipid in the liposome ranges from about 1 mol % to about 25 mol %, the mol % of cholesterol in the liposome ranges from about 1 mol % to about 50 mol % and the mol % of non-PEGylated lipid in the liposome ranges from about 45 mol % to about 98 mol %.

In some embodiments, the lipid molar ratio of the non-PEGylated neutral lipid:cholesterol:PEGylated lipid is about 9.8:5.7:0.8 or about 18:5.5:3. In some embodiments, the liposome further comprises at least one TLR agonist, optionally at least two TLR agonists. In some embodiments. the at least one TLR agonist comprises a hydrophobic tail. In some embodiments, the at least one TLR agonist is 3M-052 or GLA. In some embodiments, the TLR agonist comprises a synthetic GLA of formula:

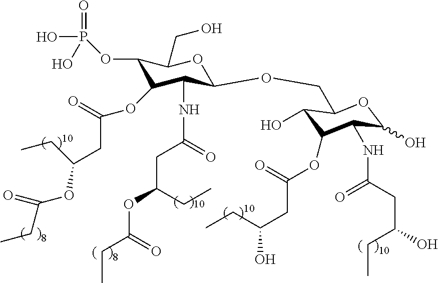

or a pharmaceutically acceptable salt thereof and/or a synthetic GLA of formula:

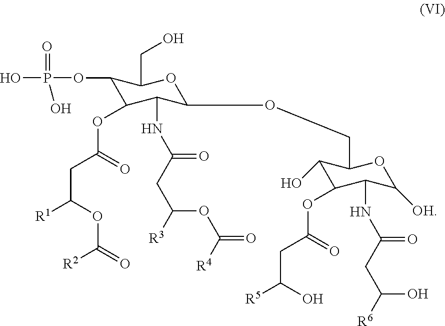

or a pharmaceutically acceptable salt thereof, wherein R¹, R³, R⁵ and R⁶ are C₁₁-C₂₀ alkyl; and R² and R⁴ are C₁₃-C₂₀ alkyl. In some embodiments, R¹, R³, R⁵ and R⁶ are C₁₁ alkyl; and R² and R⁴ are C₁₃ alkyl. In some embodiments, the TLR agonist comprises synthetic GLAs of both formulae noted herein above. In some embodiments, the liposome comprises a TLR4 agonist and a TLR7/8 agonist. In some embodiments, the liposome comprises GLA and 3M-052.

In some embodiments, the SARS-CoV-2 antigen is a Spike protein antigen, optionally a spike protein antigen comprising, consisting essentially of, or consisting of SEQ ID NO: 3 or an antigenic fragment thereof.

In some embodiments, the composition further comprises, consists essentially of, or consists of a pharmaceutically acceptable carrier, excipient, and/or diluent. In some embodiments, the pharmaceutically acceptable carrier, excipient, and/or diluent is pharmaceutically acceptable for use in a mammal, In some embodiments, the pharmaceutically acceptable carrier, excipient, and/or diluent is pharmaceutically acceptable for use in a human.

In some embodiments, the composition is a vaccine.

In some embodiments, the composition is in a thermostable lyophilized form.

The presently disclosed subject matter also relates in some embodiments to methods for using the presently disclosed compositions for stimulating anti-SARS-CoV-2 immune responses in subjects. In some embodiments, the methods comprise, consist essentially of, or consist of administering to the subject a composition as disclosed herein in an amount and via a route sufficient to stimulate an anti-SARS-CoV-2 immune response in the subject.

The presently disclosed subject matter also relates in some embodiments to methods for using the presently disclosed compositions for inducing anti-SARS-CoV-2 Th1 responses in subjects. In some embodiments, the methods comprise, consist essentially of, or consist of administering to the subject a composition as disclosed herein in an amount and via a route sufficient to stimulate an anti-SARS-CoV-2 Th1 immune response in the subject.

The presently disclosed subject matter also relates in some embodiments to methods for using the presently disclosed compositions for stimulating systemic immune responses and/or mucosal immune responses in subjects. In some embodiments, the methods comprise, consist essentially of, or consist of administering a composition as disclosed herein to the subject. In some embodiments, the administering is intranasally.

The presently disclosed subject matter also relates in some embodiments to methods for using the presently disclosed compositions for inducing anti-SARS-CoV-2 IgA responses in subjects. In some embodiments, the methods comprise, consist essentially of, or consist of administering to the subject a composition as disclosed herein in an amount and via a route sufficient to stimulate an anti-SARS-CoV-2 IgA immune response in the subject.

The presently disclosed subject matter also relates in some embodiments to methods for using the presently disclosed compositions for reducing SARS-CoV-2-induced lung injuries in subjects. In some embodiments, the methods comprise, consist essentially of, or consist of administering to the subject a composition as disclosed herein in an amount and via a route sufficient to reduce SARS-CoV-2-induced lung injury in the subject.

The presently disclosed subject matter also relates in some embodiments to methods for using the presently disclosed compositions for inducing anti-SARS-CoV-2 neutralizing antibodies in subjects. In some embodiments, the methods comprise, consist essentially of, or consist of administering to the subject a composition as disclosed herein in an amount and via a route sufficient to induce anti-SARS-CoV-2 neutralizing antibodies in the subject.

In some embodiments of the presently disclosed methods, the administering step is repeated at least one, optionally at least twice.

In some embodiments of the presently disclosed methods, at least one instance of the administering step is via an intranasal route.

In some embodiments of the presently disclosed methods, the administering step is repeated at least twice, with at least one instance of the administering step being via an intranasal route and at least one instance of the administering step being via a subcutaneous and/or intramuscular route of administration.

In some embodiments, the presently disclosed methods further comprise, consist essentially of, or consist of administering at least one booster, wherein the booster comprises, consists essentially of, or consists of administering to a subject a further dose of one or more of the compositions disclosed herein.

In some embodiments, the presently disclosed methods further comprise, consist essentially of, or consist of administering at least one additional adjuvant to a subject.

The presently disclosed subject matter also relates in some embodiments to use of a composition as disclosed herein for stimulating anti-SARS-CoV-2 immune responses, for inducing anti-SARS-CoV-2 Th1 responses, for stimulating systemic immune responses and/or mucosal immune responses, for inducing anti-SARS-CoV-2 IgA responses, for reducing SARS-CoV-2-induced lung injuries, and/or for inducing anti-SARS-CoV-2 neutralizing antibodies in subjects in need thereof.

The presently disclosed subject matter also relates in some embodiments to use of a composition as disclosed herein for the preparation of a medicament for stimulating anti-SARS-CoV-2 immune responses, for inducing anti-SARS-CoV-2 Th1 responses, for stimulating systemic immune responses and/or mucosal immune responses, for inducing anti-SARS-CoV-2 IgA responses, for reducing SARS-CoV-2-induced lung injuries, and/or for inducing anti-SARS-CoV-2 neutralizing antibodies in subjects in need thereof.

Thus, it is an object of the presently disclosed subject matter to provide compositions and methods for inducing anti-SARS-CoV-2 immune responses and for treating the consequences thereof in subjects in need thereof.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1. Spike purification. Purified full length spike protein was run on SDS-PAGE gel and silver stained. Lane 1: molecular weight marker. Lane 2: purified full length spike protein.

FIGS. 2A-2C. Adjuvanted Spike vaccine protected against SARS-CoV-2 lethal challenge and elicited neutralizing antibody response. K18-hACE2 mice (n=10 per group; control—circles; vaccinated—squares) were immunized on days 0, 14, and 28 with adjuvanted full-length spike protein using a combination of subcutaneous (SC, days 0 and 28) and intranasal (IN, day 14) routes. Mice were challenged with SARS-CoV-2 two weeks after the final immunization. FIG. 2A is a graph of survival analysis. Nine out of ten control mice succumbed to infection by day-6 post-challenge. All the vaccinated mice survived until the termination of the experiment (day-18). p≤0.0001. FIG. 2B is a graph of plasma anti-S1 IgG in control (black circles) and vaccinated (gray circles) mice. Pre-challenge plasma was collected a week after final immunization and anti-S1 IgG levels were measured by ELISA. FIG. 2C is a graph of pseudovirus entry neutralization. Pre-challenge plasma obtained a week post-third immunization was evaluated to assess the ability to inhibit pseudovirus entry in Vero E6 cells. Plasma from immunized mice (Vac-1 to Vac-3) showed neutralization capability equivalent to human convalescent plasma (conv. plasma). Boxes denote mean with standard deviation. Asterisk indicates that the results are statistically significant as follows: ***p<0.001.

FIGS. 3A-3H. Two immunizations were sufficient to protect mice from lung pathology and elicited a robust IgA response. K18-hACE2 mice were immunized with adjuvanted Spike vaccine using a subcutaneous prime (day-0) and intranasal boost (day-21). Mice were challenged 2-weeks post-second immunization and lungs were sampled on the day of the maximal clinical score (day-6) requiring animal euthanasia in the control group, an equal number of vaccinated mice were also euthanized. Representative H & E stained histology sections: Control mice (FIG. 3A) showed severe lung injury with marked infiltration of immune cells whereas immunized mice (FIG. 3B) showed attenuated lung injury closer to lung histology in a normal mouse (FIG. 3C). Immunohistochemistry: Lungs were stained with SARS-CoV-2 anti-nucleocapsid antibodies. Control mice (FIG. 3D) showed presence of virus (arrows) whereas vaccinated mice (FIG. 3E) showed clear lungs. Lung injury score. Histology sections were scored in a blinded manner (FIG. 3F). IgA ELISAs: Plasma (FIG. 3G) and bronchoalveolar lavage fluid (FIG. 3H) samples were harvested a week after the second immunization and anti-S1 response was tested using ELISA. Boxes denote mean with standard deviation. Scale bar=100 μm. Asterisks indicate that the results are statistically significant as follows: *p<0.05; **p<0.01.

FIG. 4 is a graph showing IFN-γ response. K18-hACE2 mice were immunized with adjuvanted Spike vaccine using a two-dose regimen consisting of subcutaneous prime (day-0) and intranasal boost (day-21). Samples were collected a week after final immunization. Splenocytes were re-stimulated with 10 μg/ml Spike antigen for 72 hours. IFN-γ was measured in culture supernatant using cytokine bead assay. Box denotes mean with standard deviation. PMA: Phorbol Myristate Acetate stimulation as a positive control.

### DETAILED DESCRIPTION

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the term “subject” refers to an individual (e.g., human, animal, or other organism) to be assessed, evaluated, and/or treated by the methods or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, the terms “effective amount” and “therapeutically effective amount” are used interchangeably and refer to the amount that provides a therapeutic effect, e.g., an amount of a composition that is effective to treat or prevent pathological conditions in a subject.

As used herein, the term “adjuvant” as used herein refers to an agent which enhances the pharmaceutical effect of another agent.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, a “derivative” of a compound refers to a chemical compound that can be produced from another compound of similar structure in one or more steps, such as in replacement of H by an alkyl, acyl, or amino group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “modulate”, as used herein, refers to changing the level of an activity, function, or process. The term “modulate” encompasses both inhibiting and stimulating an activity, function, or process.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

### II. Compositions of the Presently Disclosed Subject Matter

II.A. SARS-CoV-2 Antigens and Antigenic Fragments

In some embodiments, the presently disclosed subject matter relates to compositions for eliciting anti-SARS-CoV-2 immune responses in subjects in need thereof. In some embodiments, the compositions comprise, consist essentially of, or consist of a SARS-CoV-2 antigen and a liposomal adjuvant.

As used herein, the phrase “SARS-CoV-2 antigen” refers to any antigen encoded by and/or otherwise derivable from a SARS-CoV-2 genome, gene product, virus particle, and/or a cell in which the virus particle is present or had been present that, when administered to a subject, induces a cellular and/or a humoral anti-SARS-CoV-2 immune response in the subject. Exemplary SARS-CoV-2 antigen include the SARS-CoV-2 Spike protein as well as immunogenic fragments and derivatives thereof.

In some embodiments, the SARS-CoV-2 antigen comprises, consists essentially of, or consists of a Spike protein antigen. SARS-CoV-2 Spike protein antigens can include the full length Spike protein, including but not limited to a Spike protein having the amino acid sequence disclosed as Accession No. YP_009724390.1 (surface glycoprotein [Severe acute respiratory syndrome coronavirus 2]) of the GENBANK® biosequence database. This amino acid sequence corresponds to SEQ ID NO: 2.

In some embodiments, the Spike protein amino acid sequence is a modified amino acid sequence as compared to that disclosed as SEQ ID NO: 2. In some embodiments, the modified Spike protein amino acid sequence comprises, consists essentially of, or consists of SEQ ID NO: 2 with the modifications disclosed herein (e.g., one or more of an N-terminal signal peptide derived from μ-phosphatase, a “SGAG” substitution at the furin cleavage site (residues 701-704), proline substitutions at residues 1005 and 1006, and a C-terminal extension that includes one or more of a TEV cleavage site, a C-terminal T4 fibritin trimerization motif, and an 8× His Tag). Such a modified Spike protein amino acid sequence is set forth in SEQ ID NO: 3.

Accordingly, in some embodiments the SARS-CoV-2 antigen comprises, consists essentially of, or consists of the amino acid sequence as set forth in SEQ ID NO: 3.

In some embodiments, the composition is a vaccine. The antigen or immunogen, used interchangeably herein, can be used to elicit an immune response against a pathogen, as in developing a prophylactic vaccine. The immunogen can be used to elicit an immune response against a pathogen, as in developing a therapeutic vaccine, for example to treat a chronic infectious disease, including chronic viral diseases. One example would be a coronavirus, for example SARS-CoV-2, in a coronavirus-infected patient (such as but not limited to a patient with COVID-19). In some embodiments, the antigen or immunogen can be an immunogenic fragment of a virus spike (S) protein.

The immunogen can be used to elicit the rapid production of antibodies in animals for the purposes of producing antibodies. These can be, for example, custom polyclonal antibodies, obtained directly from various species used to make custom polyclonal antibodies, such as rabbits, goats, sheep, horses, cows, and camelidae. The antibodies can be obtained from serum or from colostrum.

The immunogen can be used to immunize animals (e.g. mice, but also other species, including rabbits) to accelerate the production of monoclonal antibodies, since the first step in making a monoclonal antibody is to immunize an animal so that it makes antibodies, so that its spleen cells can be fused with myeloma cells to make a hybridoma. Such monoclonal antibodies can be used in all the analytic, diagnostic, and therapeutic ways in which monoclonal antibodies are typically used.

In some embodiments, the composition (e.g., the vaccine) can further comprise a pharmaceutically acceptable carrier, excipient, and/or diluent. In some embodiments, the pharmaceutically acceptable carrier, excipient, and/or diluent is pharmaceutically acceptable for use in a mammal, optionally a human. More information with respect to formulations can be found herein below.

II.B. Liposomes and Liposomal Compositions

In some embodiments, the composition comprises the SARS-CoV-2 antigen provided with a liposomal adjuvant. In some embodiments, the liposomal adjuvant is one described in U.S. Patent Application Publication No. 2020/0138715, which is incorporated herein by reference in its entirety.

As such, in some embodiments the liposomal adjuvant is a PEGylated liposomal adjuvant. In some embodiments, the PEGylated liposomal adjuvant comprises, consists essentially of, or consists of a cholesterol; a non-PEGylated neutral lipid; and a PEGylated lipid, wherein the average molecular weight of the PEG in the PEGylated lipid is about 5000 Daltons or less. In some embodiments, the average molecular weight of the PEG in the PEGylated lipid ranges from about 750 Daltons to about 5000 Daltons, optionally about from about 750 Daltons to 2000 Daltons. In some embodiments, the average molecular weight of the PEG in the PEGylated lipid is about 2000 Daltons or less.

In some embodiments, the lipid molar ratio of the non-PEGylated neutral lipid:cholesterol:PEGylated lipid in the PEGylated liposome of the invention is about 9.8:5.7:0.8.

In some embodiments, the lipid molar ratio of the non-PEGylated neutral lipid:cholesterol:PEGylated lipid in the PEGylated liposome of the invention is about 18:5.5:3.

In some embodiments, the lipid molar ratio of the non-PEGylated neutral lipid DPPC:cholesterol:PEGylated lipid DPPE-PEG750 in the PEGylated liposome of the invention is about 9.8:5.7:0.8.

In some embodiments, the lipid molar ratio of the non-PEGylated neutral lipid DPPC:cholesterol:PEGylated lipid DPPE-PEG2000 in the PEGylated liposome of the invention is about 9.8:5.7:0.8.

In some embodiments, the lipid molar ratio of the non-PEGylated neutral lipid DPPC:cholesterol:PEGylated lipid DPPE-PEG750 in the PEGylated liposome of the invention is about 18:5.5:3.

In some embodiments, the PEGylated liposome comprises GLA, a cholesterol, a non-PEGylated neutral lipid, and a PEGylated lipid, wherein the PEG component is PEG2000.

In some embodiments, the PEGylated liposome comprises GLA, a cholesterol, a non-PEGylated neutral lipid, and a PEGylated lipid, wherein the PEG component is PEG750.

In some embodiments, the PEGylated liposome comprises 3M-052, a cholesterol, a non-PEGylated neutral lipid, and a PEGylated lipid, wherein the PEG component is PEG2000.

In some embodiments, the PEGylated liposome comprises 3M-052, a cholesterol, a non-PEGylated neutral lipid, and a PEGylated lipid, wherein the PEG component is PEG 750.

In some embodiments, the PEGylated liposome comprises 3M-052 and GLA, a cholesterol, a non-PEGylated neutral lipid, and a PEGylated lipid, wherein the PEG component is PEG2000.

In some embodiments, the PEGylated liposome comprises 3M-052 and GLA, a cholesterol, a non-PEGylated neutral lipid, and a PEGylated lipid, wherein the PEG component is PEG750.

In some embodiments, the PEGylated liposome comprises a LecA antigen, 3M-052 and GLA, a cholesterol, a non-PEGylated neutral lipid, and a PEGylated lipid, wherein the PEG component is PEG2000.

In some embodiments of the liposomal adjuvant of the presently disclosed subject matter, the lipid component of the PEGylated lipid comprises a neutral lipid. In some embodiments, the lipid component of the PEGylated lipid is 1,2-Distearoyl-sn-glycero-3-phosphoethanolamine (DSPE; CAS Number 1069-79-0), 1,2-Dipalmitoyl-sn-glycero-3-phosphocholine (DPPC; CAS Number 63-89-8), 1,2-Dioleoyl-sn-Glycero-3-Phosphocholine (DOPC; CAS Number 4235-95-4), 1,2-Dilauroyl-Sn-Glycero-3-Phosphocholine (DLPC; CAS Number 18194-25-7), 1,2-Dimyristoyl-Sn-Glycero-3-Phosphocholine (DMPC; CAS Number 18194-24-6), 1,2-Distearoyl-sn-glycero-3-phosphocholine (DSPC; CAS Number 816-94-4), (1-Palmitoyl-2-oleoyl-sn-glycero-3-phosphocholine (POPC; CAS Number 26853-31-6), 1,2-Dipalmitoyl-sn-glycero-3-phosphoethanolamine (DPPE; CAS Number 923-61-5), and/or 1,2-Dimyristoyl-sn-glycero-3-phosphorylethanolamine (DMPE; CAS Number 998-07-2), as well as combinations thereof. In particular embodiments, the lipid component of the PEGylated lipid is DSPE, DPPE, or a combination thereof. In some embodiments, the non-PEGylated neutral lipid is DPPC, DOPC, DLPC, DMPC, DSPC, POPC, DPPE, and/or DMPE, or any combination thereof. In some embodiments, the non-PEGylated neutral lipid is DPPC.

Other characteristics of the liposome component of the presently disclosed compositions include in some embodiments the following:

In some embodiments, the size of the PEGylated liposome ranges from about 1 nm to 450 nm, and can be considered to be a PEGylated nanoliposome. Such nanoliposomes are amenable to manufacturing and are filter sterilizable. Furthermore, in vivo, delivery of such nanoliposomes comprising an agent (e.g., an antigen and/or an adjuvant) typically do not display, or reduce the occurrence of a depot effect. Moreover in vivo delivery of such nanoliposomes comprising an agent (e.g., an antigen and/or an adjuvant) allow for delivery to draining lymph nodes, and allow for presentation to antigen presenting cells and allow for generation of an effective Th1-based immune response. The size of the PEGylated liposome can be assessed by known techniques in the art, including but not limited to, x-ray and laser diffraction, dynamic light scattering (DLS), CryoEM, or Malvern Zetasize. In some embodiments, the size of the PEGylated liposome refers to the Z-average diameter.

In some embodiments the size of the PEGylated liposome ranges from about 50 nm to 75 nm. In some embodiments the size of the PEGylated liposome ranges from about 50 nm to 100 nm. In some embodiments the size of the PEGylated liposome ranges from about 50 nm to 150 nm. In some embodiments the size of the PEGylated liposome ranges from about 50 nm to 200 nm. In some embodiments the size of the PEGylated liposome ranges from about 50 nm to 300 nm. In some embodiments the size of the PEGylated liposome ranges from about 20 nm to 100 nm. In some embodiments the size of the PEGylated liposome ranges from about 20 nm to 50 nm. In some embodiments the size of the PEGylated liposome ranges from about 10 nm to 200 nm. In some embodiments the size of the PEGylated liposome ranges from about 10 nm to 100 nm. In some embodiments the size of the PEGylated liposome ranges from about 10 nm to 50 nm. In some embodiments the size of the PEGylated liposome is about 1 nm, is about 5 nm, is about 10 nm, is about 15 nm, is about 20 nm, is about 25 nm, is about 30 nm, is about 35 nm, is about 40 nm, is about 45 nm, is about 50 nm, is about 55 nm, is about 60 nm, is about 65 nm, is about 70 nm, is about 75 nm, is about 80 nm, is about 85 nm, is about 90 nm, is about 95 nm, is about 100 nm, is about 105 nm, is about 110 nm, is about 115 nm, is about 120 nm, is about 125 nm, is about 130 nm, is about 135 nm, is about 140 nm, is about 145 nm, is about 150 nm, is about 155 nm, is about 160 nm, is about 165 nm, is about 170 nm, is about 175 nm, is about 180 nm, is about 185 nm, is about 190 nm, is about 195 nm, or is about 200 nm. In some embodiments, the size of the PEGylated liposome is no greater than about 1 nm, no greater than about 5 nm, no greater than about 10 nm, no greater than about 15 nm, no greater than about 20 nm, no greater than about 25 nm, no greater than about 30 nm, no greater than about 35 nm, no greater than about 40 nm, no greater than about 45 nm, no greater than about 50 nm, no greater than about 55 nm, no greater than about 60 nm, no greater than about 65 nm, no greater than about 70 nm, no greater than about 75 nm, no greater than about 80 nm, no greater than about 85 nm, no greater than about 90 nm, no greater than about 95 nm, no greater than about 100 nm, no greater than about 105 nm, no greater than about 110 nm, no greater than about 115 nm, no greater than about 120 nm, no greater than about 125 nm, no greater than about 130 nm, no greater than about 135 nm, no greater than about 140 nm, no greater than about 145 nm, no greater than about 150 nm, no greater than about 155 nm, no greater than about 160 nm, no greater than about 165 nm, no greater than about 170 nm, no greater than about 175 nm, no greater than about 180 nm, no greater than about 185 nm, no greater than about 190 nm, no greater than about 195 nm, or no greater than about 199 nm.

The PEGylated liposomes provided herein are stable, allowing for ease of use, manufacturability, transportability, and storage. The inventors have discovered that PEGylating a liposome contributes to the stability of the liposome. The physiochemical characteristics of the PEGylated liposome, including, but not limited to its size, is maintained over time, at various temperatures, and under various conditions.

In some embodiments, the PEGylated liposome exhibits reduced aggregation, or no aggregation, when compared to liposome in the absence of a PEGylated lipid. In some embodiments, the PEGylated liposome or composition comprised of PEGylated liposomes does not aggregate, displays little to no aggregation, displays reduced aggregation, or does not demonstrate an overall increase in average size over time compared to its initial size.

The stability of the PEGylated liposome can be measured by techniques familiar to those of skill in the art. In some embodiments, the stability is observed visually. Visual inspection can include inspection for particulates, flocculence, or aggregates. In some embodiments, the stability is determined by the size of the PEGylated liposome, and optionally expressed as change in size over time, or at various temperatures, or under certain conditions. In some embodiments, the stability is determined by assessing the % aggregation of PEGylated liposomes in the composition. In some embodiments, the stability is assessed by the ability of the PEGylated liposome to pass through a filter of a particular size, for example through a 0.20, 0.22 or 0.45 micron filter. In some embodiments, stability is determined by pH. In some embodiments, stability is determined by measurement of the polydispersity index (PdI), for example with the use of the dynamic light scattering (DLS) technique.

In some embodiments, the Z-average diameter of the PEGylated liposome increases less than 50%, less than 40%, less than 30%, less than 25%, less than 20%, less than 15%, less than 12%, less than 10%, less than 7%, less than 5%, less than 3%, less than 1% over the time period assayed.

In some embodiments, the PEGylated liposome is stable at 0-8° C. In some embodiments, the PEGylated liposome is stable at 0° C., 1° C., 2° C., 3° C., 4° C., 5° C., 6° C., 7° C., or 8° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years. In one exemplary embodiment, the PEGylated liposome is stable for at least 1 month at a temperature of about 2° C. to about 8° C. In another exemplary embodiment, the PEGylated liposome is stable for at least 1 month at a temperature of about 4° C. to about 8° C. In another exemplary embodiment, the PEGylated liposome is stable for at least 6 months at a temperature of about 4° C. to about 8° C. In another exemplary embodiment, the PEGylated liposome is stable for at least 1 year at a temperature of about 4° C. to about 8° C.

In some embodiments, the PEGylated liposome is stable at 8-20° C. In some embodiments, the PEGylated liposome is stable at 8-20° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years. In one exemplary embodiment, the PEGylated liposome is stable for at least 1 month at a temperature of about 8° C. to about 20° C. In another exemplary embodiment, the PEGylated liposome is stable for at least 1 month at a temperature of about 8° C. to about 20° C. In another exemplary embodiment, the PEGylated liposome is stable for at least 6 months at a temperature of about 8° C. to about 20° C. In another exemplary embodiment, the PEGylated liposome is stable for at least 1 year at a temperature of about 8° C. to about 20° C.

In some embodiments, the PEGylated liposome is stable at 20-30° C. In some embodiments, the PEGylated liposome is stable at 25° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years. In one exemplary embodiment, the PEGylated liposome is stable for at least 1 month at a temperature of about 25° C. In another exemplary embodiment, the PEGylated liposome is stable for at least 6 months at a temperature of about 25° C. In another exemplary embodiment, the PEGylated liposome is stable for at least 1 year at a temperature of about 25° C.

In some embodiments, the PEGylated liposome is stable at 30-40° C. In some embodiments, the PEGylated liposome is stable at 30° C., 31° C., 32° C., 33° C., 34° C. 35° C., 36° C., 37° C., 38° C., 39° C., or 40° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years. In one exemplary embodiment, the PEGylated liposome is stable for at least 1 month at a temperature of about 37° C.

In some embodiments, the PEGylated liposome is stable at 40-62° C. In some embodiments, the PEGylated liposome is stable at 40-62° C. for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month.

In some embodiments, the PEGylated liposome is stable at 4-8° C. for at least one year. In some embodiments, the PEGylated liposome is stable at 25° C. for at least one year.

In some embodiments, the PEGylated liposome is stable after 1-5 freeze thaws. In some embodiments, the PEGylated liposome is stable after 1, after 2, after 3, after 4, or after 5 freeze thaws.

In some embodiments, the polydispersity index of the PEGylated liposome is maintained at about 0.3 or less. In some embodiments the polydispersity index of the PEGylated liposome is maintained at about 0.25 or less. In some embodiments the polydispersity index of the PEGylated liposome is maintained at about 0.2 or less.

In some embodiments, the molar percentage (mol %) of the PEGylated lipid in the liposome ranges from about 1 mol % to about 25 mol %, the mol % of cholesterol in the liposome ranges from about 1 mol % to about 50 mol % and the mol % of non-PEGylated lipid in the liposome ranges from about 45 mol % to about 98 mol %, or any combination thereof. In some embodiments, the lipid molar ratio of the non-PEGylated neutral lipid:cholesterol:PEGylated lipid is about 9.8:5.7:0.8 or about 18:5.5:3.

In some embodiments, the liposome further comprises at least one TLR agonist, optionally at least two TLR agonists. In some embodiments, the at least one TLR agonist comprises a hydrophobic tail. In some embodiments, the at least one TLR agonist is 3M-052 (N-[4-(4-amino-2-butylimidazo[4,5-c]quinolin-1-yl)oxybutyl]octadecanamide; also called Telratolimod and MEDI9197; CAS Number 1359993-59-1; see Zhao et al., 2014) or Glucopyranosyl Lipid A (GLA; see e.g., Pantel et al., 2012), or a combination thereof.

In some embodiments, the TLR agonist comprises a synthetic GLA of formula:

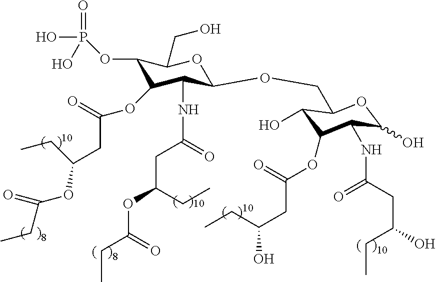

or a pharmaceutically acceptable salt thereof; a synthetic GLA of formula:

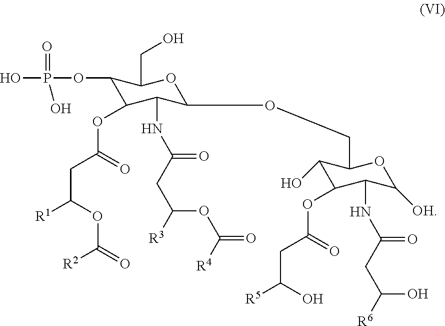

or a pharmaceutically acceptable salt thereof, wherein R¹, R³, R⁵ and R⁶ are C₁₁-C₂₀ alkyl; and R² and R⁴ are C₁₃-C₂₀ alkyl, optionally wherein R³, R⁵ and R⁶ are C₁₁ alkyl; and R² and R⁴ are C₁₃ alkyl; or any combination thereof.

In some embodiments, the liposome comprises a TLR4 agonist and a TLR7/8 agonist. Exemplary TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in U.S. Patent Application Publication No. 2010/0143301, U.S. Patent Application Publication No. 2011/0098248, and U.S. Patent Application Publication No. 2009/0047249 (Gilead Sciences, Inc., Foster City, California, United States of America), each of which is incorporated by reference in its entirety.

Examples of TLR8 modulators include motolimod, resiquimod, 3M-051, 3M-052, MCT-465, IMO-4200, VTX-763, VTX-1463, and the compounds disclosed in U.S. Patent Application Publication No. 2014/0045849 (Janssen Pharmaceuticals, Inc., Titusville, New Jersey, United States of America), U.S. Patent Application Publication No. 2014/0073642 (Janssen), PCT International Patent Application Publication No. WO 2014/056953 (Janssen), PCT International Patent Application Publication No. WO 2014/076221 (Janssen), PCT International Patent Application Publication No. WO 2014/128189 (Janssen), U.S. Patent Application Publication No. 2014/0350031 (Janssen), PCT International Patent Application Publication No. WO 2014/023813 (Janssen), U.S. Patent Application Publication No. 2008/0234251 (Array BioPharma Inc., Boulder, Colorado, United States of America), U.S. Patent Application Publication No. 2008/0306050 (Array Biopharma), U.S. Patent Application Publication No. 2010/0029585 (VentiRx Pharmaceuticals, Seattle, Washington, United States of America), U.S. Patent Application Publication No. 2011/0092485 (VentiRx Pharmaceuticals), U.S. Patent Application Publication No. 2011/0118235 (VentiRx Pharmaceuticals), U.S. Patent Application Publication No. 2012/0082658 (VentiRx Pharmaceuticals), U.S. Patent Application Publication No. 2012/0219615 (VentiRx Pharmaceuticals), U.S. Patent Application Publication No. 2014/0066432 (VentiRx Pharmaceuticals), U.S. Patent Application Publication No. 2014/0088085 (VentiRx Pharmaceuticals), U.S. Patent Application Publication No. 2014/0275167 (Novira Therapeutics LLC, Doylestown, Pennsylvania, United States of America), and U.S. Patent Application Publication No. 2013/0251673 (Novira Therapeutics), any of which can be employed alone or in combination in the compositions and methods of the presently disclosed subject matter. In some embodiments, the liposome comprises GLA and 3M-052.

In some embodiments, a liposome of the presently disclosed subject matter is prepared using techniques that are disclosed in U.S. Patent Application Publication No. 2020/0138715, which is incorporated by reference in its entirety.

II.C. Formulations

Compositions as described herein comprise in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In some embodiments, a formulation of the presently disclosed subject matter comprises an adjuvant, optionally an oil-based adjuvant.

The compositions used in the methods can take such forms as suspensions, solutions, or emulsions in oily or aqueous vehicles, and can contain formulatory agents such as suspending, stabilizing, and/or dispersing agents. The compositions used in the methods can take forms including, but not limited to perioral, intravenous, intraperitoneal, intramuscular, and intratumoral formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups, or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration, the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in oils that are administered as water-in-oil emulsions, oil-in-water emulsions, or water-in-oil-in water emulsions.

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a composition that is pharmaceutically acceptable for use in humans. One of ordinary skill in the art understands the nature of those components that can be present in such a composition that is pharmaceutically acceptable for use in humans and also what components should be excluded from compositions that are pharmaceutically acceptable for use in humans.

II.D. Doses

As used herein, the phrases “treatment effective amount”, “therapeutically effective amount”, “treatment amount”, and “effective amount” are used interchangeably and refer to an amount of a therapeutic composition sufficient to produce a measurable response (e.g., a biologically or clinically relevant response in a subject being treated). Actual dosage levels of active ingredients in the pharmaceutical compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, the condition and prior medical history of the subject being treated, etc. However, it is within the skill of the art to start doses of the compound at levels lower than required to achieve the desired therapeutic effect and to gradually increase the dosage until the desired effect is achieved.

The potency of a therapeutic composition can vary, and therefore a “therapeutically effective amount” can vary. However, one skilled in the art can readily assess the potency and efficacy of a candidate modulator of the presently disclosed subject matter and adjust the therapeutic regimen accordingly.

After review of the disclosure herein of the presently disclosed subject matter, one of ordinary skill in the art can tailor the dosages to an individual subject, taking into account the particular formulation, method of administration to be used with the composition, and other factors. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

For administration of a composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg×37 kg/m²=3700 mg/m².

For additional guidance regarding formulations and doses, see U.S. Pat. Nos. 5,326,902; 5,234,933; PCT International Publication No. WO 93/25521; Remington et al., 1975; Goodman et al., 1996; Berkow et al., 1997; Speight et al., 1997; Ebadi, 1998; Duch et al., 1998; Katzung, 2001; Gerbino, 2005.

II.E. Routes of Administration

The presently disclosed compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see, e.g., U.S. Pat. No. 6,180,082). See also U.S. Pat. Nos. 3,598,122; 5,016,652; 5,935,975; 6,106,856; 6,162,459; 6,495,605; and 6,582,724; and U.S. Patent Application Publication No. 2006/0188558 for transdermal formulations and methods of delivery of compositions. In some embodiments, the administering is via a route selected from the group consisting of peroral, intravenous, intraperitoneal, inhalation, and intratumoral.

The particular mode of administration of the compositions of the presently disclosed subject matter used in accordance with the methods disclosed herein can depend on various factors, including but not limited to the formulation employed, the severity of the condition to be treated, whether the active agents in the compositions (e.g., an anti-fibrotic) are intended to act locally or systemically, and mechanisms for metabolism or removal of the active agents following administration.

### III. Methods and Uses of the Presently Disclosed Subject Matter

The compositions of the presently disclosed subject matter can be employed as part of various uses, including but not limited to methods of treatment.

In some embodiments, the presently disclosed subject matter relates to methods for stimulating anti-SARS-CoV-2 immune responses in subjects in need thereof. In some embodiments, the methods comprise, consist essentially of, or consist of administering to the subject a composition as disclosed herein in an amount and via a route sufficient to stimulate an anti-SARS-CoV-2 immune response in the subject.

In some embodiments, the presently disclosed subject matter relates to methods for inducing anti-SARS-CoV-2 Th1 responses in subjects. In some embodiments, the methods comprise, consist essentially of, or consist of administering to the subject a composition as disclosed herein in an amount and via a route sufficient to stimulate an anti-SARS-CoV-2 Th1 immune response in the subject.

In some embodiments, the presently disclosed subject matter relates to methods for stimulating a systemic immune response and/or a mucosal immune response in a subject, optionally both a systemic immune response and a mucosal immune response in a subject. In some embodiments, the methods comprise, consist essentially of, or consist of administering a composition as disclosed herein to the subject, wherein the administering stimulates a systemic immune response, a mucosal immune response, or both in the subject. In some embodiments, the composition is administered intranasally.

In some embodiments, the presently disclosed subject matter relates to methods for inducing anti-SARS-CoV-2 IgA responses in subjects. In some embodiments, the methods comprise, consist essentially of, or consist of administering to the subject a composition as disclosed herein in an amount and via a route sufficient to stimulate an anti-SARS-CoV-2 IgA immune response in the subject.

In some embodiments, the presently disclosed subject matter relates to methods for reducing SARS-CoV-2-induced lung injuries in subjects. In some embodiments, the methods comprise, consist essentially of, or consist of administering to the subject a composition as disclosed herein in an amount and via a route sufficient to reduce SARS-CoV-2-induced lung injury in the subject.

In some embodiments, the presently disclosed subject matter relates to methods for inducing anti-SARS-CoV-2 neutralizing antibodies in subjects. In some embodiments, the methods comprise, consist essentially of, or consist of administering to the subject a composition as disclosed herein in an amount and via a route sufficient to induce anti-SARS-CoV-2 neutralizing antibodies in the subject.

In some embodiments of any of the presently disclosed methods, the administering step can be repeated at least one (e.g., a further inoculation and/or “boost”). As such, the administering step can be performed in some embodiments once, in some embodiments twice, in some embodiments three times, in some embodiments four times, in some embodiments five times, or more.

In some embodiments in which more than one administering step is performed, each administering step can be via the same route of administration or different administering steps can be via different routes of administration. In some embodiments, at least one instance of the administering step is via an intranasal route. By way of example and not limitation, in some embodiments the administering step is repeated at least twice, with at least one instance of the administering step being via an intranasal route and at least one instance of the administering step being via a subcutaneous and/or intramuscular route of administration.

In some embodiments, the composition(s) that is/are administered in each administering step is the same (e.g., a composition comprising the SARS-CoV-2 antigen comprising the amino acid sequence of SEQ ID NO: 3 and a liposome comprising a cholesterol, a non-PEGylated neutral lipid, and a PEGylated lipid as disclosed herein. In some embodiments, one or more of the different administrations employ a different composition. By way of example and not limitation, in some embodiments the first administration employs a composition comprising the SARS-CoV-2 antigen comprising the amino acid sequence of SEQ ID NO: 3 and a liposome comprising a cholesterol, a non-PEGylated neutral lipid, and a PEGylated lipid as disclosed herein, and one or more of the subsequent administrations employ a composition comprising a different SARS-CoV-2 antigen (e.g., a SARS-CoV-2 comprising an amino acid sequence that differs from SEQ ID NO: 3 by one or more amino acids) and/or a different liposome and/or adjuvant.

In some embodiments, the presently disclosed methods further comprise administering at least one booster, wherein the booster comprises, consists essentially of, or consists of administering to the subject a further dose of a composition as disclosed herein. In some embodiments, the booster employs the same composition as the initial administration. In some embodiments, the booster employs a different composition from that employed for the initial administration.

In some embodiments, the presently disclosed methods further comprise administering at least one additional adjuvant and/or co-adjuvant to the subject. Exemplary, non-limiting adjuvants and co-adjuvants and that can be employed with the compositions and methods of the presently disclosed subject matter include a retinoic acid (RA) co-adjuvant, AS-2, monophosphoryl lipid A, 3-de-O-acylated monophosphoryl lipid A, IFA, QS21, CWS, TOM, AGPs, CpG-containing oligonucleotides, Toll-like receptor (TLR) agonists, Leif, saponins, saponin mimetics, biological and synthetic lipid A, imiquimod, gardiquimod, resiquimod, polyl:C, flagellin, GLA, SLA, Stingin, and combinations thereof.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the EXAMPLES

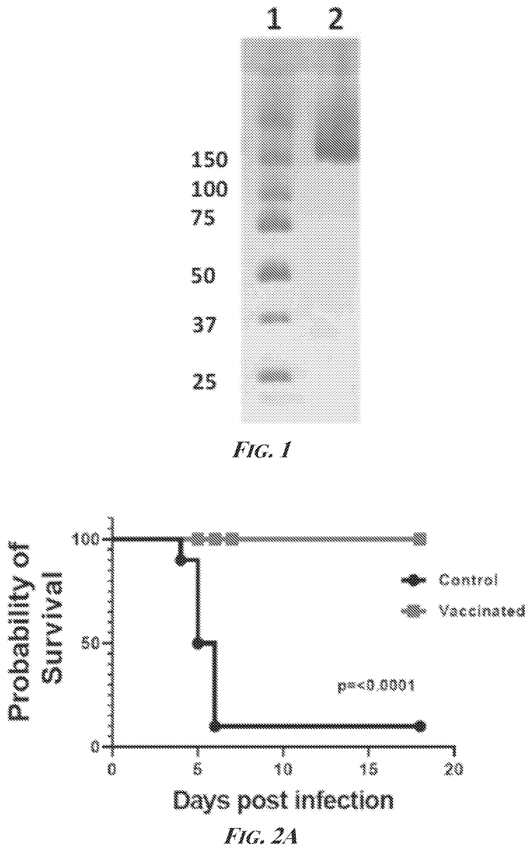

Expression and purification of SARS-CoV-2 spike protein. The SARS-CoV-2 prefusion spike ectodomain protein was recombinantly expressed in mammalian EXPICHO-SA™ cells (ThermoFisher, Waltham, Massachusetts, United States of America) using a gene encoding amino acids 1-1281 derived from Accession No. MN908947 (severe acute respiratory syndrome coronavirus 2 isolate Wuhan-Hu-1, complete genome) of the GENBANK® biosequence database (Walls et al., 2020; Wrapp et al., 2020). Basically, the construct contains an N-terminal signal peptide derived from μ-phosphatase, a “SGAG” substitution at the furin cleavage site (residues 701-704), proline substitutions at residues 1005 and 1006, and a C-terminal extension that contains a TEV cleavage site, a C-terminal T4 fibritin trimerization motif, and an 8× HisTag. The signal peptide and amino acid sequence are identical to those in Chain A (PDB code: 6VYB_A; SEQ ID NO: 1 of the SARS-CoV-2 spike ectodomain structure in the open state. Cryo-EM studies have demonstrated that this ectodomain undergoes trimerization (Walls et al., 2020). The DNA was synthesized by GeneArt (Regensburg, Germany) with codon optimization for expression in CHO cells and cloned into the mammalian expression vector pcDNA3.4 (Thermo Fisher Scientific, Waltham, Massachusetts, United States of America). The plasmid was quantified by UV spectroscopy and the sequence was confirmed by Eurofins Genomics (Louisville, Kentucky, United States of America). Transfection-grade plasmids were prepared using PURELINK™ MidiPrep kit (Thermo Fisher Scientific, Waltham, Massachusetts, United States of America). Plasmids were sterilized by filtration through a 0.2 μm spin filter (Corning, Tewksbury, Massachusetts, United States of America). Plasmid DNA was combined with EXPIFECTIMINE™ (Thermo Fisher, Massachusetts, United States of America) and transfected into EXPICHOS™ cells (Thermo Fisher, Waltham, Massachusetts, United States of America). Post-transfection, cells were cultured continuously for 13 days at 37° C., 8% CO₂ for secretion of the spike protein into the cell supernatant. The cell supernatant was collected, centrifuged at 2000×g for 30 minutes, and filtered through a 0.2 μm vacuum filter (Corning, Tewksbury, Massachusetts, United States of America). Recombinant SARS-CoV-2 spike protein expressed by CHO cells was purified using an AKTA Pure system (Cytivia, Marlborough, Massachusetts, United States of America). Talon resin (Cytivia, Marlborough, Massachusetts, United States of America) was washed with 20 column volumes of 50 mM sodium phosphate, 300 mM NaCl, 5 mM imidazole, pH 7.4. Elution was done with 50 mM sodium phosphate, 300 mM NaCl, 150 mM imidazole, pH 7.4 in 2 mL fractions using a fraction collector. Fractions containing detectable spike protein, by measuring absorbance at 280 nm, were combined and dialyzed into PBS using a Float-A-Lyzer G2 dialysis device (Repligen Corporation, Rancho Dominguez, California, United States of America). The recombinant SARS-CoV-2 spike protein was soluble in phosphate-buffered saline (PBS). The purity of the SARS-CoV-2 spike protein was assessed by silver-stained SDS-PAGE (FIG. 1) and mass spectrometry (>95% of unique peptide signal intensity). Purified spike protein was prepared for LC/MS analysis by trypsin digestion. Peptide measurements were performed using ddMS\hat{ }2 data acquisition on a Q Exactive Hybrid Quadrupole Orbitrap combined with a Vanquish Flex UHPLC system (Thermo Fisher, Waltham, Massachusetts, United States of America). Peptide mapping of spectra was performed with Sequest HT node using Proteome Discover 2.2 software (Thermo Fisher, Waltham, Massachusetts, United States of America). Endotoxin levels were assessed by Endosafe Limulus Amebocyte Lysate Assay (Charles River Laboratories Worcester, Massachusetts, United States of America).

Adjuvant formulation. GLA (Avanti Polar Lipids), 3M-052 (3M Company), dipalmitoylphosphatidylcholine (DPPC, Lipoid), PEGylated dipalmitoyl phosphatidylethanolamine (mPEG2000-DPPE, Corden), cholesterol (Sigma), and α-tocopherol (Spectrum Chemical) were mixed in chloroform. Following evaporation of the organic solvent by overnight rotary evaporation, the lipid film was hydrated with 25 mM ammonium phosphate buffer, pH 5.8, to achieve concentrations of 1 mg/ml GLA, 0.4 mg/ml 3M-052, 3.4 mg/ml DPPC, 1 mg/ml mPEG2000-DPPE, 1 mg/ml cholesterol, and 0.09 mg/ml α-tocopherol. The mixture was sonicated for about 1 hour in a water bath at 60° C. The formulation was then processed at 18,000 psi for 5 cycles on a model LM20 microfluidizer (Microfluidics Corp.), followed by filtration through a 0.8/0.2-μm membrane and storage in glass vials at 2-8° C. The adjuvant formulation was mixed at 1:1 volume with antigen and saline diluent at the time of immunization, resulting in an administered adjuvant dose of 10 μg GLA and 4 μg 3M-052.

Immunoassay to assess antibody binding to the spike 51 domain. A flat bottom 96-well clear polystyrene plate (Nunc, Rochester, New York, United States of America) was coated with rSARS-CoV-2 S1 domain (Wuhan Hu-1, amino acids 14 to 685, GeneScript, New Jersey) at 5 μg/ml in coating buffer (50 mM Carbonate/Bicarbonate pH 9.6) and incubated overnight at 4° C. The wells were washed three times with PBS-0.1% Tween 20 (PBS-T) and blocked with 100 μl/well of PBS-T containing 1% bovine serum albumin (PBS-T 1% BSA) by incubating for 1 hour at room temperature. Dilutions of the test (immunized) and control (nonimmunized) mice plasma were prepared in PBS-T 1% BSA. As a standard, immune plasma was combined in a pool. After washing the blocked plate three times with PBS-T, each plasma dilution was dispensed at 100 μl/well. After incubation for 1 hour at room temperature, the plate was washed again as above. The secondary antibody, peroxidase-conjugated affinity-purified goat anti-mouse IgG F(ab′)2 (Catalogue #115-036-006, Jackson ImmunoResearch Labs, West Grove, Pennsylvania, United States of America), was prepared at 1:2000 dilution in PBS-T 1% BSA, dispensed at 100 μl/well, and incubated at room temperature for 1 hour. The plate was then washed three times with PBS-T, and the ABTS (2,2′-Azinobis [3-ethylbenzothiazoline-6-sulfonic acid]-diammonium salt) substrate (1 mM ABTS in 70 mM citrate phosphate buffer, pH 4.2, containing 0.1% hydrogen peroxide) was dispensed at 100 μl/well. The plate was incubated at room temperature until the green color was developed and the absorbance of the lowest dilutions (1:100) at 405 nM reached between 2.0 and 2.5. The absorbance at each well was then read at 405 nm. For IgA ELISAs, a two-fold diluted plasma or undiluted BAL fluid samples were used to check for the presence of anti-S1 antibodies. Anti-mouse IgA-HRP (Catalogue #1040-05, SouthernBiotech) at 1:5000 dilution prepared in PBS-T 1% BSA was used as a secondary antibody and absorbance was read at 450 nm after addition of TMB peroxidase substrate (Seracare).

Immunizations. K18 hACE-2 transgenic C57/B6 male mice were purchased from the Jackson Laboratories. Six to ten weeks old mice were immunized subcutaneously (neck region) or intranasally under anesthesia with either 2-weeks interval (three immunization regimen) or 3-weeks interval (two immunization regimen) as mentioned in the figure legends. Typically, each mouse received 10 μg antigen that was mixed with adjuvants (10 μg GLA and 4 μg 3M-052 in liposomes) just prior to immunization. An intranasal dose consisted of 10 μl per nare whereas the subcutaneous dose was 100 μl in the neck region per animal. A 2-week interval was based on our previous studies using these adjuvants. A 3-week interval in the second experiment was based on the actual interval recommended in humans for commercial vaccines. Regimen selection was based upon our work on the amebiasis vaccine (Abhyankar et al., 2018). For the immunogenicity experiments, tissues were harvested from a subset of control and vaccinated mice a week after the final immunization.

Viral propagation. SARS-CoV-2 isolate Hong Kong/VM20001061/2020 (NR-52282) was obtained from the Biodefense and Emerging Infections Research Resources Repository (BEI Resources), National Institute of Allergy and Infectious Diseases (NIAID), National Institutes of Health (NIH). Virus was propagated in Vero C1008, Clone E6 (ATCC CRL-1586) cells cultured in Dulbecco's Modified Eagle's Medium (DMEM, Gibco 11995040) supplemented with 10% fetal bovine serum (FBS) and grown at 37° C., 5% CO₂.

Challenge. K18 hACE-2 transgenic mice were immunized either two or three times as described. Mice were challenged intranasally 2 weeks after final immunization under ketamine/xylazine anesthesia with 1300 Plaque Forming Units (PFUs) of SARS-CoV-2 (Passage-2) in 50 μl. Mice were observed daily for clinical signs. Categories included in clinical scoring included weight loss, posture, and appearance of fur (piloerection), activity, and eye closure. All mouse work was approved by the Institutional Animal Care and Use Committee of the University of Virginia (Charlottesville, Virginia, United States of America), and all procedures were performed in a certified animal Biosafety Level Three laboratory at the University of Virginia.

IFN-γ measurement. Splenocytes were counted after RBC lysis and a total of 2×10⁵ splenocytes/well in 200 μl were stimulated with Spike antigen at 20 μg/ml or left unstimulated in a 96 well U-bottom plate for 72 hours at 37° C., 5% CO₂. Supernatants were banked at −20° C. Supernatants were analyzed for secreted IFN-γ by cytokine bead array according to the manufacturer's instructions (Bio-Techne). The signal from splenocytes stimulated with PMA-ionomycin served as a positive control and the signal from blank well-containing medium alone served as a negative control.

Histology. Tissues were fixed in formalin. Slides were scanned at 20× magnification. Hematoxylin and eosin (H&E) stained histology sections were scored in a blinded manner using the guidelines of the American Thoracic Society (Matute-Bello et al., 2011) and included the following parameters-neutrophils in the alveolar space, neutrophils in the interstitial space, hyaline membranes, proteinaceous debris filling the air spaces, and alveolar septal thickening. Lungs from age-matched mice served as a negative control.

Immunohistochemistry. Slides of fixed lung tissue were stained with SARS-CoV-2 specific anti-nucleoprotein antibody (Catalogue #9099, ProSci, Poway, California, United States of America) as per manufacturer's instructions at the University of Virginia's biorepository and tissue research core facility. Slides were scanned at 20× magnification.

Pseudovirus neutralization assay. Antibody potency was assessed by standard pseudovirus neutralization assays, in which the concentration of antiserum required to prevent cell infection by non-replicative pseudotyped SARS-CoV-2 virus is determined. Assays were performed as described in Sengar et al., 2021. Briefly, pseudovirus was generated using a plasmid encoding SARS-CoV-2 S protein (Wuhan-Hu-1) with the 19 C-terminal amino acids removed, a MLV Gag-Pol helper plasmid, and a plasmid encoding a luciferase gene containing a packaging sequence (the latter two gifts of Judith White, University of Virginia, Charlottesville, Virginia, United States of America). Approximately 1 million HEK 293 T/17 cells were transfected with 11 μg plasmid and 3 μL Lipofectamine 2000 transfection reagent (Thermo Fisher Scientific, Waltham, Massachusetts, United States of America). The supernatant containing pseudovirus particles was collected after 48 hours, clarified for cell debris at 700×g for 7 minutes at 4° C., filtered through a 0.45 μm PES syringe filter, stored at 4° C., and used within 24 hours. Infection with pseudovirus causes luciferase expression and was quantitatively assayed via luminescence 72 hours after infection, via which IC₅₀ values for antibody inhibition of cellular infection were calculated. Plasma samples were diluted to the specified ratio, and combined with a standardized amount of pseudovirus, and incubated for 1 hour at 37° C. to permit antibody binding before addition to approximately 125,000 Vero E6 cells per well of a 24-well plate. Plates were centrifuged for 30 minutes at 100×g, 4° C. for pseudovirus attachment and then incubated at 37° C., 5% CO₂. After 6 hours, 400 μl complete media was added to each well. Luciferase activity was measured in a plate reader 60-78 hours post-infection using the BRITELITE™ reagent (PerkinElmer Inc., Waltham, Massachusetts, United States of America). Uncertainty quantification was performed via bootstrap resampling. Discarded plasma sample collected on day-32 post-COVID-19 detection from University of Virginia Medical Center inpatient was used as a source of convalescent plasma (IRB-HSR #22231 and 200110).

Statistics. GraphPad Prism, Matlab, and Microsoft Excel were used to generate the Figures A two-tailed Student's t test was used to determine statistical significance for IgG ELISAs. Mann-Whitney test was used to calculate statistical significance between the groups for IgA ELISAs and clinical scores. Logrank Mantel-Cox test was used to determined statistical significance for survival analysis. Histological scoring was analyzed using a two-tailed Student's t test.

### Example 1

### Adjuvanted Spike Vaccine Protected Against Lethal Infection

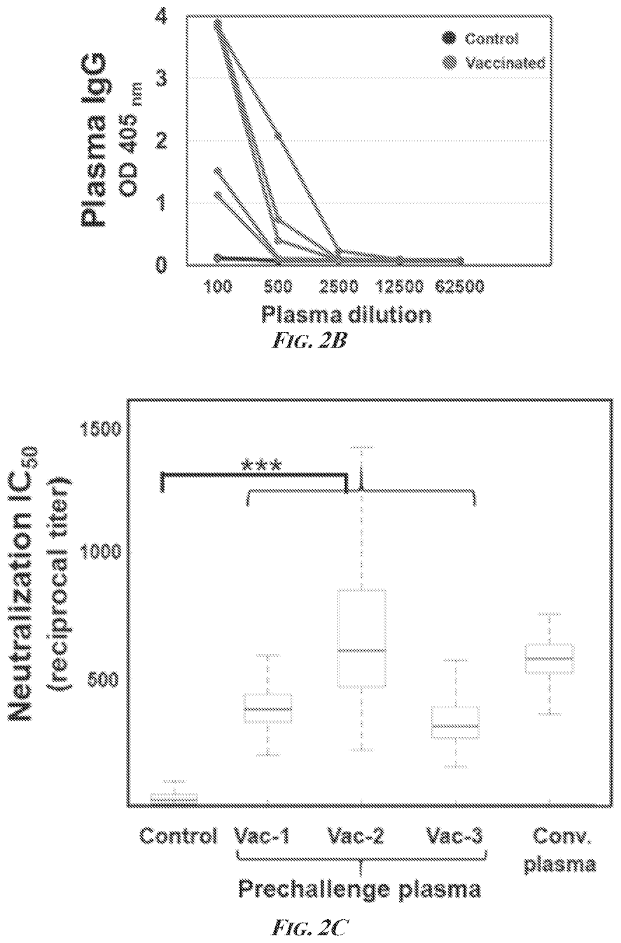

The protective efficacy of GLA 3M-052-liposomes adjuvanted full-length spike antigen was evaluated in a mouse model of SARS-CoV-2 infection (Moreau et al., 2020). K18-hACE2 mice, (n=10 per group), were immunized three times with adjuvanted spike vaccine using a mixed regimen comprising of a subcutaneous prime followed by an intranasal and a subcutaneous boost. A 2-week interval was maintained between each immunization (Abhyankar et al., 2018; Abhyankar et al., 2021b). The antigen and adjuvant doses for the immunizations were based on the design of experiments statistical approach (Abhyankar et al., 2021a). Mice in the control group received only adjuvant. All the mice were challenged 2 weeks after the third immunization intranasally with 1300 pfu SARS-CoV-2. Nine of ten control group mice succumbed to infection by day-6 post-challenge, whereas all the mice from the vaccinated group survived (FIG. 2A) and displayed no clinical signs for the duration of the experiment (19 days post-challenge). Pre-challenge plasma collected a week after final immunization showed high anti-Spike IgG titer (FIG. 2B) that effectively neutralized entry of spike pseudotyped virus into Vero cells with an IC₅₀ value that was equivalent to convalescent plasma from COVID-19 patient (FIG. 2C).

### Example 2

### Two Immunizations Protected Against Lung Immunopathology

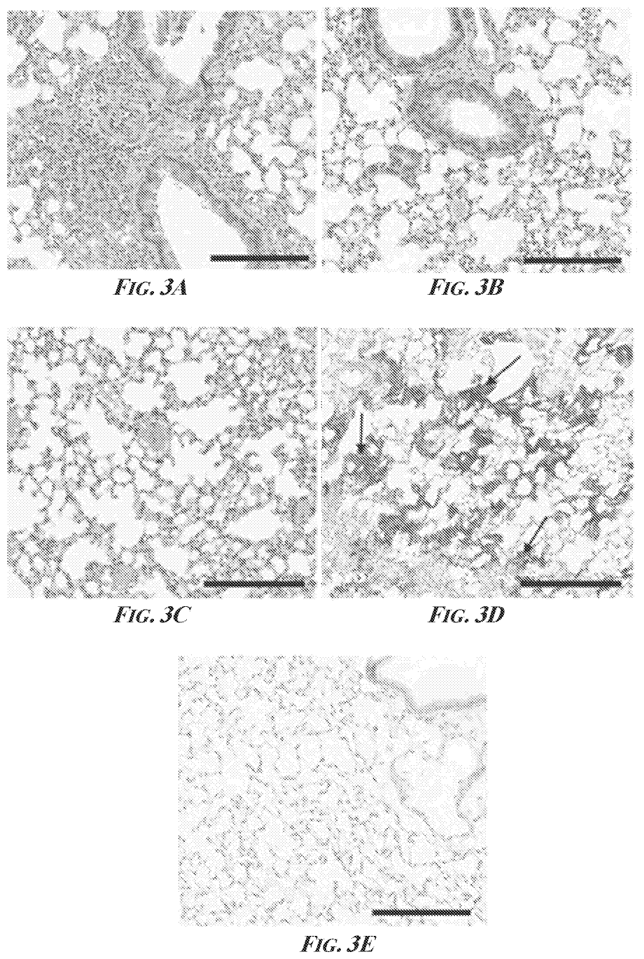

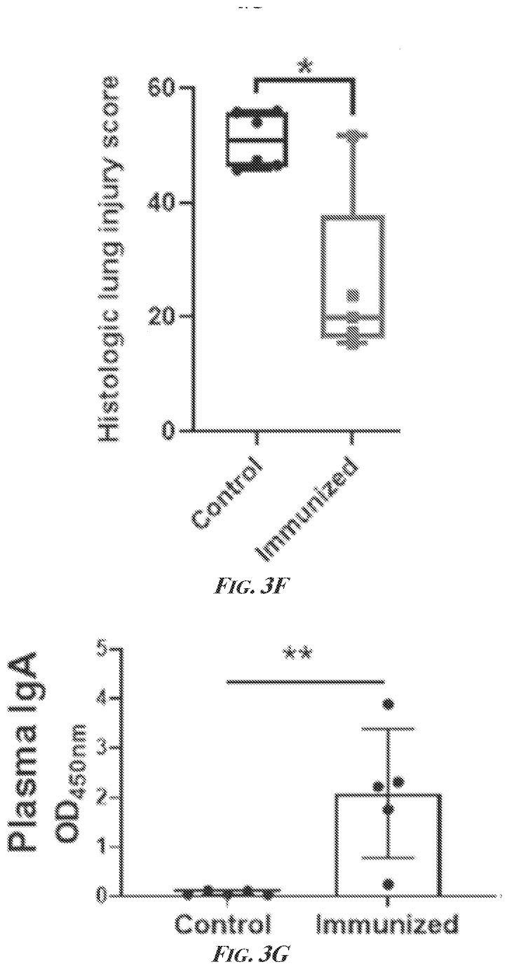

To determine whether two immunizations were sufficient to protect mice from lung immunopathology, K18-hACE2 mice (n=10 per group) were immunized using a subcutaneous prime and intranasal boost. A three-week interval was maintained be two immunizations to mimic the current immunization regimen for the commercially approved COVID-19 vaccines. Mice were challenged two weeks post-second immunization and the experiment was terminated on day-8 post-challenge. At this time point, vaccinated mice did not exhibit any clinical signs upon challenge (mean clinical score 0.7±0.5), in contrast to the control mice, which showed higher clinical scores (mean clinical score 3.7±1.9, p=0.002). While six of the 10 mice in the control group succumbed to infection by day 8, all the vaccinated mice survived. Lungs obtained from the control mice during peak infection revealed multifocal inflammatory infiltrates (FIG. 3A) whereas vaccinated mice showed significantly reduced lung injury (FIG. 3B) more similar to uninfected mice (FIG. 3C). Control mice also revealed significantly higher viral load as seen after staining with anti-nucleocapsid antibodies (FIG. 3D) as compared to vaccinated mice (FIG. 3E). Histologic injury scoring revealed significantly higher scores for the control mice versus the vaccinated mice (FIG. 3F).

Finally, the ability of the experimental vaccine to elicit local and systemic SARS-CoV-2 S-specific IgA response was evaluated in the two immunization regimen. Robust IgA levels were detected in plasma (FIG. 3G) and bronchoalveolar lavage (BAL) fluid (FIG. 3H) in the mice vaccinated using subcutaneous prime-intranasal boost regimen, whereas control mice failed to show any anti-Spike IgA response.

### Discussion of the EXAMPLES

The most important outcome of this work was a demonstration of the rapid development of a highly efficacious COVID-19 vaccine using a dual TLR ligand liposome adjuvant. This adjuvant platform was recently characterized for its ability to concurrently develop a strong mucosal, as well as a Th1 type systemic immune response using amebiasis vaccine as a model (Abhyankar et al., 2018). Adjuvanted spike vaccine-induced robust systemic neutralizing antibodies and completely protected mice from a lethal SARS-CoV-2 challenge. The lung histology from vaccinated mice showed relative protection from the robust lung injury seen in the control mice. These observations were also reflected in the lung viral loads. Thus, this fully synthetic liposomal adjuvant administered with SARS-CoV-2 spike antigen showed an excellent protection efficacy in a mouse model of COVID-19.

COVID-19 patients show highly variable plasma neutralizing antibody response (Robbiani et al., 2020) and data about the durability of neutralizing antibodies is unclear (Isho et al., 2020; Sariol & Perlman, 2020). An effective vaccine should be able to elicit high levels of neutralizing antibodies to the SARS-CoV-2 spike protein and TLR based adjuvants have been shown to enhance effective neutralizing antibody titers against many viruses (Duthie et al., 2011; Martinsen et al., 2020). The inclusion of TLR ligands in adjuvanted subunit COVID vaccine elicited a Th1 biased response in monkeys compared to those without TLR ligands in a recent study and a Th1 biased response may be beneficial against COVID-19 (Arunachalam et al., 2021; Gil-Etayo et al., 2021). The presently disclosed adjuvant platform elicited a robust anti-Spike neutralizing antibody response which was comparable to that seen in convalescent plasma from COVID-19 patients.

Another important observation was the demonstration that two doses were sufficient to prevent lung injury in vaccinated mice. Control mice consistently showed significantly higher clinical scores compared to vaccinated mice. Histologically, vaccinated mice showed reduced infiltration of immune cells into the alveolar interstitium throughout the course of the experiment and no detectable virus upon staining with virus-specific antibodies. Control mice, on the other hand, consistently showed lung infiltration of immune cells by histology, as well as high viral burden by immunohistochemistry.

Coordinated local mucosal and systemic immune responses are important for protection against COVID-19 pathologies. An ideal COVID-19 vaccine should be able to generate a strong mucosal as well as systemic response in both the humoral and cellular immune compartments. Immunization in this study elicited an anti-Spike IgA response in plasma as well as BAL fluid, confirming the potential of the current adjuvant combination to elicit antigen-specific mucosal IgA response (Abhyankar et al., 2021b). Several recent studies have emphasized the importance of SARS-CoV-2 specific IgA antibodies, especially within the respiratory system, in providing effective immunity (Ejemel et al., 2020). Specifically, dimeric IgA which is a secretory form at mucosal surfaces was 15-fold more potent than their monomeric counterparts (Wang et al., 2021). Sterlin et al., 2021 showed that IgA contributed to virus neutralization to a greater extent compared with IgG in an infected cohort of patients. Moreover, although IgA serum concentrations decreased one month after the onset of symptoms, neutralizing mucosal IgA remained detectable in saliva for a longer time. Thus, mucosal anti-Spike IgA response appears to be a critical feature of an effective vaccine as it can play a crucial role in barrier function at the first port of viral entry. GLA 3M-052 liposomes were efficient in generating antigen-specific mucosal IgA response, especially when used via mucosal regimen (Abhyankar et al., 2018) and this might be one of the reasons for the 100% protective efficacy disclosed herein.

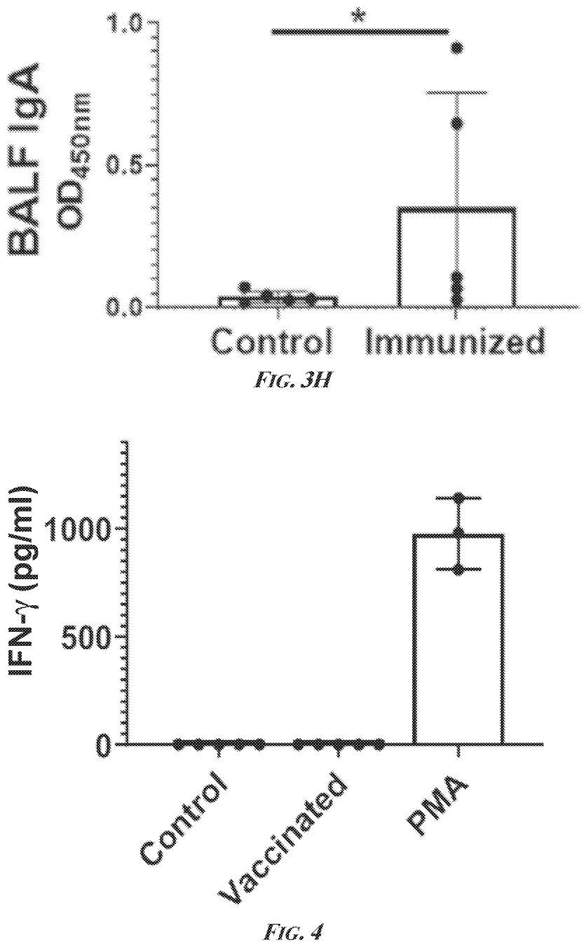

The present disclosure indicated that although subcutaneous prime-intranasal booster combination elicited local and systemic IgA response, interestingly, splenocytes from these mice did not produce detectable IFN-γ upon re-stimulation with the antigen (FIG. 4). We have not checked IFN-γ levels in the BAL fluid. It is known that intranasal vaccination is capable of stimulating immune responses in the nasopharynx-associated lymphoid tissue and the respiratory tract in addition to systemic locations (Wang et al., 2020). Intranasal immunization with RBD domain of the S protein in MERS-CoV was shown to elicit a more powerful local mucosal immune system in the lung tissue in comparison with the subcutaneous immunization (Ma et al., 2014). This also supports our previous observations that intranasal immunization helps induce a strong mucosal immune response compared to parenteral routes. This intriguing observation needs further characterization.

Together, these data demonstrated that this synthetic adjuvant system with pharmacologically acceptable components elicited a robust antigen-specific humoral response against an RNA virus and fully protected mice from clinical disease. While we have not characterized cellular immune responses, our previous studies indicated a strong antigen-specific T cell response when using GLA 3M-052 liposomes. Development of new COVID-19 vaccines with equivalent efficacies to currently approved vaccines remains essential with special focus on cross protection, the durability of the immune response, ease of administration, stability, as well as cost. For the present and future, emerging or re-emerging pandemic diseases, there will be an overwhelming demand for cost-effective vaccines, which can be developed rapidly. The availability of an adjuvant that has been tested in pre-clinical and clinical trials can address most of these requirements. Overall, GLA 3M-052 Liposome is a promising “plug-and-play” adjuvant platform for the rapid development of effective and safe vaccines.

```
EXEMPLARY SEQUENCES
Chain A, Spike glycoprotein (PDB 6VYB_A; SEQ ID NO: 1)
      1 mgilpspgmp allslvslls vllmgcvaet gtqcvnlttr tqlppaytns ftrgvyypdk
    61 vfrssvlhst qdlflpffsn vtwfhaihvs gtngtkrfdn pvlpfndgvy fasteksnii
  121 rgwifgttld sktqsllivn natnvvikvc efqfcndpfl gvyyhknnks wmesefrvys
  181 sannctfeyv sqpflmdleg kqgnfknlre fvfknidgyf kiyskhtpin lvrdlpqgfs
  241 aleplvdlpi ginitrfqtl lalhrsyltp gdsssgwtag aaayyvgylq prtfllkyne
  301 ngtitdavdc aldplsetkc tlksftvekg iyqtsnfrvq ptesivrfpn itnlcpfgev
  361 fnatrfasvy awnrkrisnc vadysvlyns asfstfkcyg vsptklndlc ftnvyadsfv
  421 irgdevrqia pgqtgkiady nyklpddftg cviawnsnnl dskvggnyny lyrlfrksnl
  481 kpferdiste iyqagstpcn gvegfncyfp lqsygfqptn gvgyqpyrvv vlsfellhap
  541 atvcgpkkst nlvknkcvnf nfngltgtgv ltesnkkflp fqqfgrdiad ttdavrdpqt
  601 leilditpcs fggvsvitpg tntsnevavl yqdvnctevp vaihadqltp twrvystgsn
  661 vfqtragcli gaehvnnsye cdipigagic asyqtqtnsp sgagsvasqs iiaytmslga
  721 ensvaysnns iaiptnftis vtteilpvsm tktsvdctmy icgdstecsn lllqygsfct
  781 qlnraltgia veqdkntqev faqvkqiykt ppikdfggfn fsqilpdpsk pskrsfiedl
  841 lfnkvtlada gfikqygdcl gdiaardlic aqkfngltvl pplltdemia qytsallagt
  901 itsgwtfgag aalqipfamq mayrfngigv tqnvlyenqk lianqfnsai gkiqdslsst
  961 asalgklqdv vnqnaqalnt lvkqlssnfg aissvlndil srldppeaev qidrlitgrl
1021 qslqtyvtqq liraaeiras anlaatkmse cvlgqskrvd fcgkgyhlms fpqsaphgvv
1081 flhvtyvpaq eknfttapai chdgkahfpr egvfvsngth wfvtqrnfye pqiittdntf
1141 vsgncdvvig ivnntvydpl qpeldsfkee ldkyfknhts pdvdlgdisg inasvvniqk
1201 eidrlnevak nlneslidlq elgkyeqyik gsgrenlyfq ggggsgyipe aprdgqayvr
1261 kdgewvllst flghhhhhhh h
GENBANK ® Accession No. YP_0097243901.1: surface glycoprotern
[Severe acute respiratory syndrome coronavirus 2] (SEQ ID NO: 2)
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIK
Modified surface glycoprotein from Severe acute respiratory
syndrome coronavirus 2 with enhanced stability (SEQ ID NO: 3)
QCVNLTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEF
QFCNDPFLGVYYHKNNKSWMESEFRVYSSANNCTFEYVSQPFLMDLEGKQGNFKNLREFV
FKNIDGYFKIYSKHTPINLVRDLPQGFSALEPLVDLPIGINITRFQTLLALHRSYLTPGD
SSSGWTAGAAAYYVGYLQPRTFLLKYNENGTITDAVDCALDPLSETKCTLKSFTVEKGIY
QTSNERVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCV
IAWNSNNLDSKVGGNYNYLYRLFRKSNLKPFERDISTEIYQAGSTPCNGVEGFNCYFPLQ
SYGFQPTNGVGYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTNTSNEVAVLYQ
DVNCTEVPVAIHADQLTPTWRVYSTGSNVFQTRAGCLIGAEHVNNSYECDIPIGAGICAS
YQTQTNSPSGAGSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYKTPP
IKDFGGFNFSQILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMAYRFNGIGVTQ
NVLYENQKLIANQFNSAIGKIQDSLSSTASALGKLQDVVNQNAQALNTLVKQLSSNFGAI
SSVLNDILSRLDPPEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPREG
VFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
```

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Abhyankar et al. (2017) Nanoformulation of synergistic TLR ligands to enhance vaccination against Entamoeba histolytica. Vaccine 35:916-922.
Abhyankar et al. (2018) Adjuvant composition and delivery route shape immune response quality and protective efficacy of a recombinant vaccine for Entamoeba histolytica. NPJ Vaccines 3:22.
Abhyankar et al. (2021a) Optimizing a multi-component intranasal Entamoeba histolytica vaccine formulation using a design of experiments strategy. Front Immunol 12:683157.
Abhyankar et al. (2021b) Development of COVID-19 vaccine using a dual Toll-like receptor ligand liposome adjuvant. npj Vaccines 18; 6(1):137
Arunachalam et al. (2021) Adjuvanting a subunit COVID-19 vaccine to induce protective immunity. Nature 594:253-258.
Berkow et al. (1997) The Merck Manual of Medical Information, Home ed. Merck Research Laboratories, Whitehouse Station, New Jersey.
Chia et al. (2021) Dynamics of SARS-CoV-2 neutralising antibody responses and duration of immunity: a longitudinal study. Lancet Microbe 2:e240-e249.
COVID-19 vaccine tracker and landscape. https://www.who.int/publications/m/item/draft-landscape-of-covid-19-candidate-vaccines.
Desai et al. (2020) Temporal and spatial heterogeneity of host response to SARS-CoV-2 pulmonary infection. Nat Commun 11:6319.
Duch et al. (1998) Volatile anesthetics significantly suppress central and peripheral mammalian sodium channels. Toxicol Lett 100-101:255-263.
Duthie et al. (2011) Use of defined TLR ligands as adjuvants within human vaccines. Immunol Rev 239:178-196.
Ebadi (1998) CRC Desk Reference of Clinical Pharmacology. CRC Press, Boca Raton, Florida, United States of America.
Ejemel et al. (2020) A cross-reactive human IgA monoclonal antibody blocks SARSCoV-2 spike-ACE2 interaction. Nat Commun 11:4198.
Freireich et al. (1966) Quantitative comparison of toxicity of anticancer agents in mouse, rat, hamster, dog, monkey and man. Cancer Chemother Rep 50:219-244.
GENBANK® Accession Nos. MN908947.3; YP_009724390.1.
Gerbino (2005) Remington: The Science and Practice of Pharmacy. 21st Edition. Lippincott Williams & Wilkins, Philadelphia, Pennsylvania, United States of America.
Gil-Etayo et al. (2021) T-helper cell subset response is a determining factor in COVID-19 progression. Front Cell Infect Microbiol 11:624483.
Global Health Estimates: Life expectancy and leading causes of death and disability. https://www.who.int/data/maternal-newborn-child-adolescent-ageing/advisorygroups/gama/activities-of-gama.
Goodman et al. (1996) Goodman & Gilman's the Pharmacological Basis of Therapeutics, 9th ed. McGraw-Hill Health Professions Division, New York.
Hodgson et al. (2021) What defines an efficacious COVID-19 vaccine? A review of the challenges assessing the clinical efficacy of vaccines against SARS-CoV-2. Lancet Infect Dis 21:e26-e35.
Huang et al. (2020) Clinical features of patients infected with 2019 novel coronavirus in Wuhan, China. Lancet 395:497-506.
Isho et al. (2020) Persistence of serum and saliva antibody responses to SARS-CoV-2 spike antigens in COVID-19 patients. Sci Immunol 5:eabe5511.
Jeyanathan et al. (2020) Immunological considerations for COVID-19 vaccine strategies. Nat Rev Immunol 20:615-632.
Katzung (2001) Basic & Clinical Pharmacology, 8th ed. Lange Medical Books/McGraw-Hill Medical Pub. Division, New York.
Luchner et al. (2021) TLR agonists as vaccine adjuvants targeting cancer and infectious diseases. Pharmaceutics 13:142.
Ma et al. (2014) Intranasal vaccination with recombinant receptor-binding domain of MERS-CoV spike protein induces much stronger local mucosal immune responses than subcutaneous immunization: implication for designing novel mucosal MERS vaccines. Vaccine 32:2100-2108.
Manson et al. (2020) COVID-19-associated hyperinflammation and escalation of patient care: a retrospective longitudinal cohort study. Lancet Rheumatol 2:e594-e602.
Martinsen et al. (2020) The use of toll-like receptor agonists in HIV-1 cure strategies. Front Immunol 11:1112.
Matute-Bello et al. (2011) An official American Thoracic Society workshop report: features and measurements of experimental acute lung injury in animals. Am J Respir Cell Mol Biol 44:725-738.
Moreau et al. (2020) Evaluation of K18-hACE2 mice as a model of SARS-CoV-2 infection. Am J Trop Med Hyg 103:1215-1219.
Pantel et al. (2012) A new synthetic TLR4 agonist, GLA, allows dendritic cells targeted with antigen to elicit Th1 T cell immunity in vivo. Eur J Immunol 42(1):101-109.
PCT International Patent Application Publication Nos. WO 93/25521, WO 2014/056953, WO 2014/076221, WO 2014/128189, WO 2014/023813, WO 2017/200957.
PDB Accession No. 6VYB_A.
Remington et al. (1975) Remington's Pharmaceutical Sciences, 15th ed. Mack Pub. Co., Easton, Pennsylvania.
Robbiani et al. (2020) Convergent antibody responses to SARS-CoV-2 in convalescent individuals. Nature 584:437-442.
Sariol & Perlman (2020) Lessons for COVID-19 immunity from other coronavirus infections. Immunity 53:248-263.
Sengar et al. (2021) Single-virus fusion measurements yield an opportunistic model for SARS-CoV-2 fusion. bioRxiv 2021.05.04.442634.
Speight et al. (1997) Avery's Drug Treatment: A Guide to the Properties, Choice, Therapeutic Use and Economic Value of Drugs in Disease Management, 4th ed. Adis International, Auckland/Philadelphia, United States of America.
Sterlin et al. (2021) IgA dominates the early neutralizing antibody response to SARSCoV-2. Sci Transl Med 13:eabd2223.
Teijaro & Farber (2021) COVID-19 vaccines: modes of immune activation and future challenges. Nat Rev Immunol 21:195-197.
U.S. Patent Application Publication Nos. 2006/0188558, 2008/0234251, 2008/0306050, 2009/0047249, 2010/0029585, 2011/0092485, 2011/0118235, 2010/0143301, 2011/0098248, 2012/0082658, 2012/0219615, 2013/0251673, 2014/0045849, 2014/0073642, 2014/0350031, 2014/0066432, 2014/0088085, 2014/0275167.
U.S. Pat. Nos. 3,598,122; 5,016,652; 5,234,933; 5,326,902; 5,935,975; 6,106,856; 6,162,459; 6,180,082; 6,495,605; 6,582,724.
Walls et al. (2020) Structure, Function, and Antigenicity of the SARS-CoV-2 Spike Glycoprotein. Cell 183:1735.
Wang et al. (2020) Emerging role of mucosal vaccine in preventing infection with Avian influenza a viruses. Viruses 12:862.
Wang et al. (2021) Enhanced SARS-CoV-2 neutralization by dimeric IgA. Sci Transl Med 13:eabf1555.
Wrapp et al. (2020) Cryo-EM structure of the 2019-nCoV spike in the prefusion conformation. Science 367:1260-1263.
Zhao et al. (2014) Combination therapy targeting toll like receptors 7, 8 and 9 eliminates large established tumors. J Immunother Cancer 2: Article No. 12.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A composition for eliciting an anti-SARS-CoV-2 immune response in a subject in need thereof, the composition comprising, consisting essentially of, or consisting of:
(i) a SARS-CoV-2 antigen; and
(ii) a PEGylated liposomal adjuvant, wherein the PEGylated liposomal adjuvant comprises, consists essentially of, or consists of:
(a) a cholesterol;
(b) a non-PEGylated neutral lipid; and
(c) a PEGylated lipid, wherein the average molecular weight of the PEG in the PEGylated lipid is about 5000 Daltons or less.

2. The composition of claim 1, wherein the average molecular weight of the PEG in the PEGylated lipid ranges from about 750 Daltons to about 5000 Daltons, optionally about from about 750 Daltons to 2000 Daltons.

3. The composition of claim 1, wherein the average molecular weight of the PEG in the PEGylated lipid is about 2000 Daltons or less.

4. The composition of claim 1, wherein the lipid component of the PEGylated lipid comprises a neutral lipid.

5. The composition of claim 1, wherein the lipid component of the PEGylated lipid is DSPE, DPPC, DOPC, DLPC, DMPC, DSPC, POPC, DPPE, or DMPE.

6. The composition of claim 1, wherein the lipid component of the PEGylated lipid is DSPE or DPPE.

7. The composition of claim 1, wherein the non-PEGylated neutral lipid is DPPC, DOPC, DLPC, DMPC, DSPC, POPC, DPPE, or DMPE.

8. The composition of claim 1, wherein the non-PEGylated neutral lipid is DPPC.

9. The composition of claim 1, wherein the liposome is stable for at least 1 month at a temperature of about 2° C. to about 8° C.

10. The composition of claim 1, wherein the polydispersity index of the liposome is maintained at about 0.3 or less.

11. The composition of claim 1, wherein the size of the liposome less than about 450 nm.

12. The composition of claim 1, wherein the molar percentage (mol %) of the PEGylated lipid in the liposome ranges from about 1 mol % to about 25 mol %, the mol % of cholesterol in the liposome ranges from about 1 mol % to about 50 mol % and the mol % of non-PEGylated lipid in the liposome ranges from about 45 mol % to about 98 mol %.

13. The composition of claim 1, wherein the lipid molar ratio of the non-PEGylated neutral lipid:cholesterol:PEGylated lipid is about 9.8:5.7:0.8 or about 18:5.5:3.

14. The composition of claim 1, wherein the liposome further comprises at least one TLR agonist, optionally at least two TLR agonists.

15. The composition of claim 14, wherein the at least one TLR agonist comprises a hydrophobic tail.

16. The composition of claim 14, wherein the at least one TLR agonist is 3M-052 or GLA.

17. The composition of claim 14, wherein the TLR agonist comprises a synthetic GLA of formula: 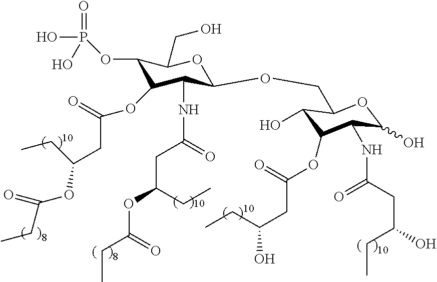
or a pharmaceutically acceptable salt thereof; a synthetic GLA of formula: 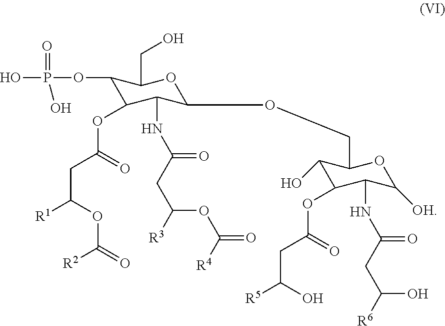
or a pharmaceutically acceptable salt thereof, wherein:
R¹, R³, R⁵ and R⁶ are C₁₁-C₂₀ alkyl; and R² and R⁴ are C₁₂-C₂₀ alkyl, optionally wherein R¹, R³, R⁵ and R⁶ are C₁₁ alkyl; and R² and R⁴ are C₁₃ alkyl;
or a combination thereof.

18. The composition of claim 14, wherein the liposome comprises a TLR4 agonist and a TLR7/8 agonist.

19. The composition of claim 18, wherein the liposome comprises GLA and 3M-052.

20. The composition of claim 1, wherein the SARS-CoV-2 antigen is a Spike protein antigen, optionally a spike protein antigen comprising, consisting essentially of, or consisting of SEQ ID NO: 3 or an antigenic fragment thereof.

21. The composition of claim 1, wherein the composition further comprises a pharmaceutically acceptable carrier, excipient, and/or diluent.

22. The composition of claim 21, wherein the pharmaceutically acceptable carrier, excipient, and/or diluent is pharmaceutically acceptable for use in a mammal, optionally a human.

23. The composition of claim 21, wherein the composition is a vaccine.

24. The composition of claim 23, wherein the composition is in a thermostable lyophilized form.

25. A method for stimulating an anti-SARS-CoV-2 immune response in a subject, the method comprising, consisting essentially of, or consisting of administering to the subject the composition of claim 1 in an amount and via a route sufficient to stimulate an anti-SARS-CoV-2 immune response in the subject.

26. A method for inducing an anti-SARS-CoV-2 Th1 response in a subject, the method comprising, consisting essentially of, or consisting of administering to the subject the composition of claim 1 in an amount and via a route sufficient to stimulate an anti-SARS-CoV-2 Th1 immune response in the subject.

27. A method for stimulating a systemic immune response and a mucosal immune response in a subject, the method comprising, consisting essentially of, or consisting of administering the composition of claim 1 to the subject, optionally wherein the administering is intranasally.

28. A method for inducing an anti-SARS-CoV-2 IgA response in a subject, the method comprising, consisting essentially of, or consisting of administering to the subject the composition of claim 1 in an amount and via a route sufficient to stimulate an anti-SARS-CoV-2 IgA immune response in the subject.

29. A method for reducing SARS-CoV-2-induced lung injury in a subject, the method comprising, consisting essentially of, or consisting of administering to the subject the composition of claim 1 in an amount and via a route sufficient to reduce SARS-CoV-2-induced lung injury in the subject.

30. A method for inducing anti-SARS-CoV-2 neutralizing antibodies in a subject, the method comprising, consisting essentially of, or consisting of administering to the subject the composition of claim 1 in an amount and via a route sufficient to induce anti-SARS-CoV-2 neutralizing antibodies in the subject.

31. The method of claim 25, wherein the administering step is repeated at least one, optionally at least twice.

32. The method of claim 31, wherein at least one instance of the administering step is via an intranasal route.

33. The method of claim 32, wherein the administering step is repeated at least twice, with at least one instance of the administering step being via an intranasal route and at least one instance of the administering step being via a subcutaneous and/or intramuscular route of administration.

34. The method of claim 32, wherein the method further comprises administering at least one booster, wherein the booster comprises, consists essentially of, or consists of administering to the subject a further dose of the composition of any one of claims 1-22.

35. The method of claim 25, further comprising administering at least one additional adjuvant to the subject.

36. Use of a composition of claim 1 for stimulating anti-SARS-CoV-2 immune responses, for inducing anti-SARS-CoV-2 Th1 responses, for stimulating systemic immune responses and/or mucosal immune responses, for inducing anti-SARS-CoV-2 IgA responses, for reducing SARS-CoV-2-induced lung injuries, and/or for inducing anti-SARS-CoV-2 neutralizing antibodies in subjects in need thereof.

37. Use of a composition of claim 1 for the preparation of a medicament for stimulating anti-SARS-CoV-2 immune responses, for inducing anti-SARS-CoV-2 Th1 responses, for stimulating systemic immune responses and/or mucosal immune responses, for inducing anti-SARS-CoV-2 IgA responses, for reducing SARS-CoV-2-induced lung injuries, and/or for inducing anti-SARS-CoV-2 neutralizing antibodies in subjects in need thereof.


SEQUENCE LISTING (3 sequences)

SEQ ID NO: 1 - protein, 1281 aa, Artificial Sequence
  Artifically synthesized spike protein sequence
MGILPSPGMPALLSLVSLLSVLLMGCVAETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNEVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGRENLYFQGGGGSGYIPEAPRDGQAYVRKDGEWVLLSTFLGHHHHHHHH

SEQ ID NO: 2 - protein, 1211 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 3 - protein, 1198 aa, Artificial Sequence
  Artifically synthesized spike protein sequence
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNEVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK